(19) United States
(12) Patent Application Publication
(10) Pub. No.: US 2023/0200869 A1
(43) Pub. Date: Jun. 29, 2023

(54) BONE ANCHORING DEVICE

(71) Applicant: MOREH COMPANY CO., LTD, Seoul (KR)

(72) Inventor: Kwang Hyun JI, Seoul (KR)

(73) Assignee: MOREH COMPANY CO., LTD, Seoul (KR)

(21) Appl. No.: 17/916,430

(22) PCT Filed: Sep. 22, 2020
(86) PCT No.: PCT/KR2020/012782

§ 371 Date: Sep. 30, 2022

(30) Foreign Application Priority Data
Apr. 3, 2020 (KR) 10-2020-0040863

(51) Int. Cl. A61B 17/80; A61B 17/86

(52) U.S. Cl. CPC A61B 17/8052; A61B 17/8605; A61B 2017/00862

(57) ABSTRACT

The present invention relates to a bone anchoring device including a plate configured to be attached to a fractured bone and having a plurality of fastening holes to support the fractured bone, in which at least one of the fastening holes is formed in a rectangular shape having an opening portion elongated in a longitudinal direction, a clip configured to be inserted into the rectangular fastening hole and capable of slipping in the longitudinal direction in the rectangular fastening hole, and an anchoring screw configured to be fastened into the clip and fastened into the bone. According to the present invention, it is possible to adjust a position and insertion angle of the bone anchoring screw and improve compatibility between bone anchoring components.

### TECHNICAL FIELD

The present invention relates to a bone anchoring device, and more particularly, to a bone anchoring device in which a rectangular fastening hole is formed in a plate configured to come into close contact with a fractured bone, a clip is inserted into the fastening hole, and the clip slips in a transverse direction in the fastening hole, such that a position of a bone anchoring screw for fixing the bone may be adjusted.

### BACKGROUND ART

In general, in case of a patient with fractures, the fractures are joined by bonding the fractures to an exact position and anchoring the fractures in an immovable state until bones at two opposite sides of the fractures coalesce. In this case, there are various methods including a representative method of attaching a plate to a bone surface and fastening and fixing screws to two opposite sides of a fractured bone.

That is, the screws are inserted into holes in the plate and fastened to the bone, thereby pressing the plate against the bone and fixing the plate.

Meanwhile, recently, various methods have been provided to fix the bone in accordance with types of fractures and diseased parts. That is, a better result may be made in respect to the joining of the fracture when a movement between fracture surfaces is permitted instead of when the fracture surfaces are not matched or the bones at the particular site are completely in close contact with each other and fixed in an immovable state. In this case, a technology for fixing the fractured bone while permitting a predetermined movement between the fracture surfaces has been developed and applied to clinical trials.

In this regard, Korean Patent Application Laid-Open No. 10-2016-0073130 discloses a bone anchoring device including: a body part (plate) configured to come into close contact with a bone and having a plurality of coupling holes and a long hole; a head part connected to one end of the body part and having a plurality of coupling holes; screws configured to be inserted into the coupling holes; and a block detachably installed in the long hole by using a sliding screw. The head part is formed to have a predetermined angle with respect to a lower surface thereof, and the other end of the body part has a predetermined angle with respect to a lower surface of the body part.

However, because the bone anchoring device is devised to anchor a bone, such as an ankle bone, at a particular site, there are problems in that the plate and the bone anchoring screw of the bone anchoring device need to be manufactured for the particular purpose, and the inherent bone anchoring screw suitable for the characteristics of the plate need to be used. For this reason, the bone anchoring device cannot be used universally.

In addition, there are problems in that the position of the position of the bone anchoring screw is not easily displaced, and a bone insertion position and an angle of the screw cannot be simultaneously adjusted.

### DISCLOSURE

### Technical Problem

The present invention has been made in an effort to solve the above-mentioned problems in the related art, and an object of the present invention is to provide a bone anchoring device that uses the same plate and enables a practitioner to freely select an anchoring position of a screw and the type of screw to be used depending on a state of a diseased part to be treated.

### Technical Solution

To achieve the above-mentioned object, one aspect of the present invention relates to a bone anchoring device, which anchors a fractured bone by fixing a plate being in close contact with a fractured bone by using a screw, the bone anchoring device including: a plate configured to be attached to the fractured bone and having a plurality of fastening holes to support the fractured bone, in which at least one of the fastening holes is formed in a rectangular shape having an opening portion elongated in a longitudinal direction; a clip configured to be inserted into the rectangular fastening hole and capable of slipping in the longitudinal direction in the rectangular fastening hole; and an anchoring screw configured to be fastened into the clip and fastened into the bone.

In the bone anchoring device according to one aspect of the present invention, the rectangular fastening hole may be formed in the longitudinal direction of the plate.

In addition, the clip may have an elastic structure having a cut-out portion, the clip may be inserted into the rectangular fastening hole while being elastically compressed, and the separation of the clip from the rectangular fastening hole may be restricted as the clip is elastically deformed after the clip is inserted into the rectangular fastening hole.

In addition, an outer peripheral surface of the clip may be formed as a curved surface convex outward, and an inner surface of the rectangular fastening hole based on the longitudinal direction may be formed as a curved surface concave inward that corresponds to the outer peripheral surface of the clip.

Further, a guide protrusion may protrude outward from the outer peripheral surface of the clip, a guide groove may be recessed inward in the inner surface of the rectangular fastening hole based on the longitudinal direction at a position corresponding to the guide protrusion, the clip may slip only in a horizontal direction, and angular deformation and a rotation may be restricted.

In addition, the guide protrusion may be formed in a polyhedral shape.

Further, an edge of the guide protrusion may be formed in a curved shape.

The clip may have anti-rotation protrusions protruding and extending outward by a predetermined length from the outer peripheral surface at two opposite ends of the cut-out portion, such that the cut-out portion of the clip may be maintained to be directed in the longitudinal direction of the rectangular fastening hole.

Further, the anti-rotation protrusion may protrude in a direction parallel to a centerline of the cut-out portion.

In addition, an outer peripheral surface of the clip may be formed as a flat surface that adjoins an inner surface of the rectangular fastening hole in the longitudinal direction, and the rectangular fastening hole may have a flat surface that is concave inward so that the inner surface based on the longitudinal direction corresponds to the outer peripheral surface of the clip.

In addition, the anchoring screw may include: a head part configured to be seated on an inner peripheral surface of the clip; and a body part extending from an inner end of the head part, configured to come into contact with the bone, and having a screw thread formed on an outer peripheral surface thereof.

Further, the head part may be configured as a motion screw having an inclined curved surface having a cross-section with a width that decreases downward, and the inner peripheral surface of the clip may have an inclined curved surface having a diameter that decreases downward while corresponding to the head part.

The head part may be configured as a locking screw having an inclined side surface having a cross-section with a width that decreases from above to below, and the inner peripheral surface of the clip may be an inclined surface having an inclination and a diameter that decreases from above to below while corresponding to the head part.

A cortex screw having cut-out grooves formed at predetermined intervals may be formed in a lateral surface of the head part.

In addition, a fixing screw thread may be formed on an outer peripheral surface of the head part and configured to be screw-coupled to the inner peripheral surface of the clip, and an inner screw thread may be formed on the inner peripheral surface of the clip while corresponding to the fixing screw thread.

Further, the screw thread of the body part may include: an insertion thread portion formed at an end of the body part; and an extraction thread portion formed between the insertion thread portion and the head part and configured to provide a drawing force to the anchoring screw by being screw-coupled to the bone and rotated in case of drawing and removing the anchoring screw.

In addition, a screw thread pitch of the extraction thread portion may be larger than a screw thread pitch of the insertion thread portion.

Further, a drill tip may be formed at an end of the insertion thread portion to drill the bone in case of inserting the anchoring screw into the bone.

### Advantageous Effects

According to the bone anchoring device according to the present invention configured as described above, the clip may be inserted into the rectangular fastening hole formed in the plate, the clip may slip in the longitudinal direction in the fastening hole, and the anchoring position of the screw may be restrictively deformed in the plate by an external force, such that the effect of joining the particular portions may be improved.

In addition, according to the present invention, various screws may be applied to the single plate without separately manufacturing the plate for the respective screws to be used, thereby improving the compatibility between the bone anchoring components.

In addition, the procedure may be performed while variably adjusting the insertion position of the screw and applying the motion screw or the cortex screw capable of changing the insertion angle.

Further, in the present invention, the clip, which is elastically inserted, is applied as a medium to ensure versatility of the screws, and the elasticity direction is constantly maintained when the clip is fixed, which makes it possible to provide a constant and stable fixing force.

### DESCRIPTION OF DRAWINGS

FIG. 1 is a perspective view of a bone anchoring device according to an embodiment of the present invention.

FIG. 2 is a perspective view of a clip used for the bone anchoring device in

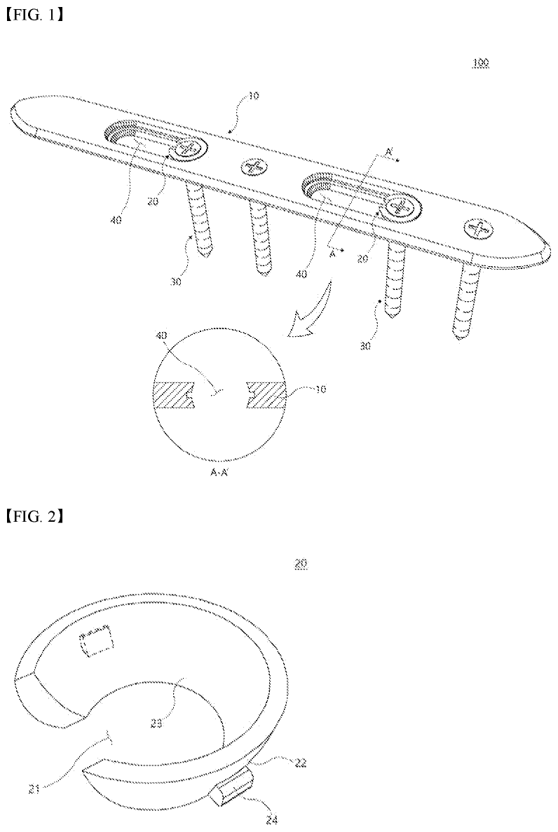

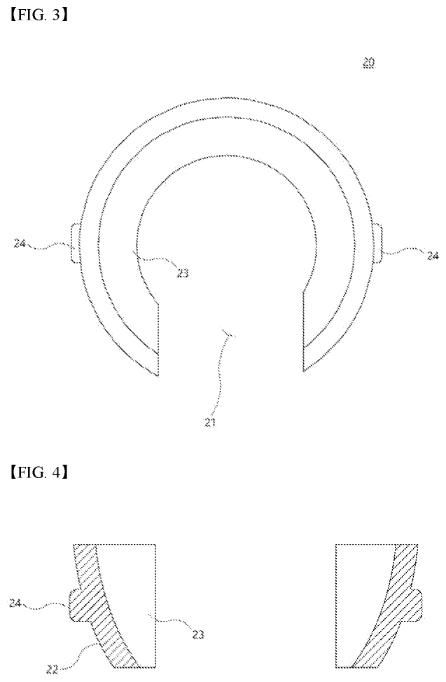

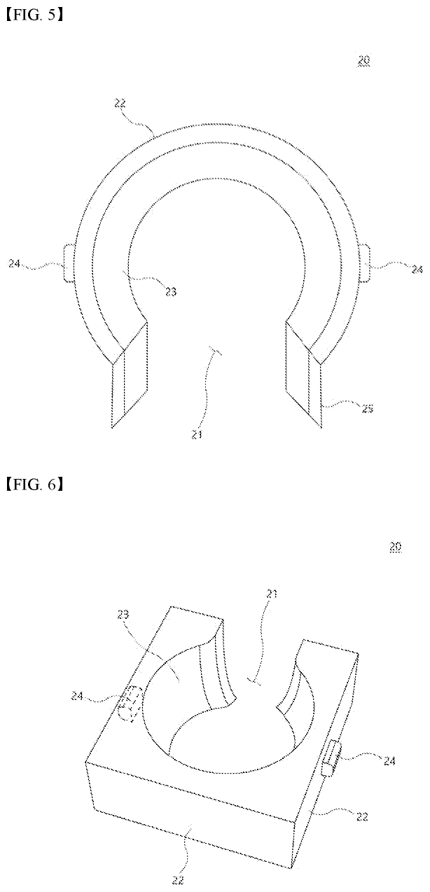

FIG. 1.

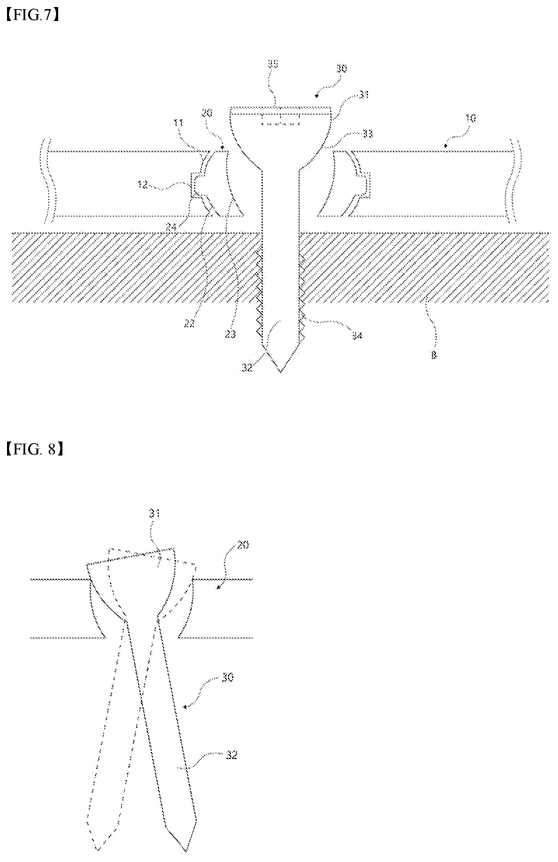

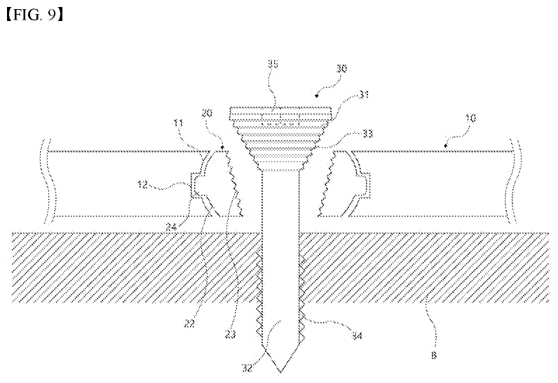

FIG. 3 is a top plan view of the clip in FIG. 2.

FIG. 4 is a cross-sectional view of the clip in FIG. 2.

FIG. 5 is a top plan view of another embodiment of the clips used for the bone anchoring device according to the present invention.

FIG. 6 is a top plan view of still another embodiment of the clip used for the bone anchoring device according to the present invention.

FIG. 7 is a cross-sectional view of an embodiment in which the bone anchoring device according to the present invention is applied.

FIG. 8 is a cross-sectional view illustrating an example in which an insertion angle of a bone anchoring screw is changed in the bone anchoring device according to the present invention.

FIG. 9 is a cross-sectional view of another embodiment in which the bone anchoring device according to the present invention is applied.

### BEST MODE

To achieve the above-mentioned object, one aspect of the present invention relates to a bone anchoring device, which anchors a fractured bone by fixing a plate being in close contact with a fractured bone by using a screw, the bone anchoring device including: a plate configured to be attached to the fractured bone and having a plurality of fastening holes to support the fractured bone, in which at least one of the fastening holes is formed in a rectangular shape having an opening portion elongated in a longitudinal direction; a clip configured to be inserted into the rectangular fastening hole and capable of slipping in the longitudinal direction in the rectangular fastening hole; and an anchoring screw configured to be fastened into the clip and fastened into the bone. The rectangular fastening hole is formed in the longitudinal direction of the plate, the clip has an elastic structure having a cut-out portion, the clip is inserted into the rectangular fastening hole while being elastically compressed, and the separation of the clip from the rectangular fastening hole is restricted as the clip is elastically deformed after the clip is inserted into the rectangular fastening hole.

In this case, an outer peripheral surface of the clip is formed as a curved surface convex outward, and an inner surface of the rectangular fastening hole based on the longitudinal direction is formed as a curved surface concave inward that corresponds to the outer peripheral surface of the clip.

Further, a guide protrusion protrudes outward from the outer peripheral surface of the clip, a guide groove is recessed inward in the inner surface of the rectangular fastening hole based on the longitudinal direction at a position corresponding to the guide protrusion, the clip slips only in a horizontal direction, and angular deformation and a rotation are restricted.

### MODES OF THE INVENTION

The present invention may be variously modified and may have various embodiments, and particular embodiments illustrated in the drawings will be described in detail below. However, the description of the exemplary embodiments is not intended to limit the present invention to the particular exemplary embodiments, but it should be understood that the present invention is to cover all modifications, equivalents and alternatives falling within the spirit and technical scope of the present invention. In the description of the present invention, the specific descriptions of publicly known related technologies will be omitted when it is determined that the specific descriptions may obscure the subject matter of the present invention.

The terms such as “first” and “second” may be used to describe various constituent elements, but the constituent elements should not be limited by the terms. These terms are used only to distinguish one constituent element from another constituent element.

The terminology used herein is used for the purpose of describing particular embodiments only and is not intended to limit the present invention. Singular expressions include plural expressions unless clearly described as different meanings in the context. The terms “comprises,” “comprising,” “includes,” “including,” “containing,” “has,” “having” or other variations thereof are inclusive and therefore specify the presence of stated features, integers, steps, operations, elements, and/or components, but do not preclude the presence or addition of one or more other features, integers, steps, operations, elements, components, and/or groups thereof.

Hereinafter, a bone anchoring device according to the present invention will be more specifically described with reference to exemplary embodiments and the accompanying drawings.

FIG. 1 is a perspective view of a bone anchoring device according to an embodiment of the present invention. Referring to FIG. 1, a bone anchoring device 100 according to the present invention includes a plate 10, clips 20, and anchoring screws 30.

In the present invention, the plate 10 serves to safely support a fractured part by being in close contact with a surface of a bone and is formed to conform to a shape of a fractured bone B. An inner surface of the plate 10 may be a curved surface that surrounds the bone.

In this case, according to the present invention, the plate 10 may have a plurality of fastening holes into which the screws for fixing the bone are inserted. At least one of the plurality of fastening holes may be formed in a rectangular shape having an opening portion elongated in a longitudinal direction. In general, the rectangular fastening hole 40 may be formed in a longitudinal direction of the plate 10. The rectangular fastening hole 40 will be described below more specifically.

In addition, in the present invention, the clip 20 is inserted into the rectangular fastening hole 40. The anchoring screw 30 is inserted into the clip 20 and serves to fix the bone by being inserted into the bone. In this regard, FIG. 2 is a perspective view of the clip 20 used for the bone anchoring device according to the present invention, FIG. 3 is a top plan view of the clip, and FIG. 4 is a cross-sectional view of the clip.

FIGS. 2, 3, and 3B, the clip 20 may be formed in an annular shape with a ring shape having a cut-out portion 21. In this case, the clip 20 may have an elastic structure made of an elastic material. Therefore, according to the present invention, when a practitioner holds an outer peripheral surface of the clip 20 with his/her hand and compresses the outer peripheral surface of the clip 20 inward, the clip 20 may be elastically compressed and inserted into the rectangular fastening hole 40. In addition, after the clip is inserted into the rectangular fastening hole 40, the clip 20 is elastically deformed and comes into close contact with an inner surface 11 of the rectangular fastening hole 40 while being elastically compressed. Therefore, the clip 20 is prevented from separating to the outside of the rectangular fastening hole 40.

To this end, according to the present invention, an outer peripheral surface 22 of the clip 20 is formed as a curved surface convex outward, and the inner surface 11 of the rectangular fastening hole 40 based on the longitudinal direction is formed as a curved surface concave inward that corresponds to the outer peripheral surface of the clip 20, such that the clip 20 is seated in the rectangular fastening hole 40 while being in close contact with the rectangular fastening hole 40. In addition, this configuration enables the clip 20 to move (slip) in the longitudinal direction along the inner surface 11 of the rectangular fastening hole 40. As a result, it is possible to adjust a position of the anchoring screw 30 inserted into the clip 20.

Therefore, according to the present invention, an anchoring position of the screw 20 is allowed to be restrictively changed by an external force in the plate 10, which makes it possible to improve an effect of joining the bones at the particular site. That is, in the case of a site such as articular bones that inevitably move even after the bones are joined, the screw 30 may move to some extent as the clip 20 slips in the rectangular fastening hole 40 of the plate 10 even after the bone is fixed by the screw 30, such that a motion of a joint portion may be allowed.

In addition, according to the present invention, a guide protrusion 23 may protrude outward from the outer peripheral surface 22 of the clip 20, and a guide groove 12 may be recessed inward in the inner surface 11 of the rectangular fastening hole 40 based on the longitudinal direction and correspond to the guide protrusion 23.

Therefore, the clip 20 inserted into the rectangular fastening hole 40 moves only in a horizontal direction, i.e., the longitudinal direction in the rectangular fastening hole 40, and a rotation of the clip 20 about a central axis in a longitudinal direction (long-side direction) of the rectangular fastening hole 40 is restricted. Therefore, the screw is prevented from being inserted into the bone at an inclination angle that is not intended by the practitioner. Therefore, the anchoring screw 30 may be stably inserted into the bone.

In this case, the guide protrusion 24 may have various shapes. Particularly, the guide protrusion 24 may have a polyhedral shape such as a hexahedral shape. An edge portion of the guide protrusion 24 is rounded, such that the guide protrusion 24 may be smoothly inserted into a guide groove 12 formed in the inner surface 11 of the rectangular fastening hole 40.

In this case, in case that the guide protrusion 24 is provided in the form of a plate-shaped member having upper and lower surfaces that are flat surfaces, the angular deformation and the rotation of the guide protrusion 24 about a central axis in a short-side direction of the rectangular fastening hole 40 (an axis perpendicular to the axis of the long-side direction) are also restricted, such that the screw may be more stably fastened.

Meanwhile, FIG. 5 is a top plan view of another embodiment of the clip 20. Referring to FIG. 5, the clip 20 may have anti-rotation protrusions 25 protruding and extending outward by a predetermined length from the outer peripheral surface at two opposite ends of the cut-out portion 21. In this case, the anti-rotation protrusion 25 may protrude in a direction parallel to a centerline of the cut-out portion 21. Therefore, according to the present invention, the clip 20 is prevented from rotating in a leftward/rightward direction in the rectangular fastening hole 40. Therefore, the cut-out portion 21 of the clip 20 may be maintained to be directed in the longitudinal direction of the rectangular fastening hole 40.

That is, according to the present invention, the guide protrusions 24 formed on the outer peripheral surface 22 of the clip 20 prevent the clip 20 from rotating in an upward/downward direction in the rectangular fastening hole 40, and the anti-rotation protrusions 25 prevent the clip 20 from rotating in the leftward/rightward direction in the rectangular fastening hole 40, such that the clip 20 stably slips only in the longitudinal direction in the rectangular fastening hole 40.

Therefore, according to the present invention, the practitioner may freely and accurately select the screw insertion position depending on a state of a diseased part.

FIG. 6 is a view of still another embodiment of the clip 20 according to the present invention. According to the present embodiment, the outer peripheral surface 22 of the clip 20, which adjoins the surface 11 of the rectangular fastening hole 40 based on the longitudinal direction may have a flat structure. In addition, the rectangular fastening hole 40 may have a flat surface that is concave inward so that the inner surface 11 based on the longitudinal direction corresponds to the outer peripheral surface 22 of the clip 20. Therefore, according to the present invention, the clip 20 may be prevented from rotating in the leftward/rightward direction in the rectangular fastening hole 40, and the clip 20 slips only in the longitudinal direction, such that the fractured bone may be stably fixed.

In addition, according to the present invention, the clip 20 may be formed such that a diameter of the inner peripheral surface 23 of the clip 20 decreases toward an upper expanded inclined surface, i.e., from above to below. Therefore, according to the present invention, the anchoring screw 30 for fixing the bone is gradually inserted into the bone from the inner peripheral surface 23 of the clip 20, an inclined surface of a head part of the screw presses the inclined surface of the inner periphery of the clip 20, and the clip 20 is elastically deformed in a transverse direction of the plate 10 comes into close contact with the inner surface 11 of the rectangular fastening hole 40, such that the anchoring position of the anchoring screw 30 is maintained.

Meanwhile, FIG. 7 is a cross-sectional view of an embodiment in which the bone anchoring device 100 according to the present invention is applied, and FIG. 8 is a cross-sectional view illustrating an example in which an insertion angle of the bone anchoring screw 30 is changed.

The plate 10 and the anchoring screw 30, which constitutes the present invention, are fastened by means of the clip 20. Therefore, when the clip is deformed in accordance with the applied screw, the clip, which is matched with various screws, may be used for the same plate.

Therefore, referring to FIGS. 7 to 8, the anchoring screw 30, which is configured to fix the bone by being inserted into the fractured bone while penetrating the clip 20, basically includes: a head part 31 seated on the inner peripheral surface 23 of the clip 20; and a body part 32 extending from an inner end of the head part 31, configured to be inserted into the bone, and having a screw thread 34 formed on an outer peripheral surface thereof.

In this case, according to the embodiment of the present invention, the head part 31 may be configured as a motion screw having an inclined curved surface having a cross-section with a width that decreases downward. Therefore, in this case, the inner peripheral surface 23 of the clip 20 may have an inclined curved surface having a diameter that decreases downward while corresponding to the head part 31. In addition, a wrench groove 35 may be formed in an upper surface of the head part 31 so that a wrench may be fitted into the wrench groove 35.

Therefore, according to the present invention, as illustrated in FIG. 7, the head part 31 may rotate relative to the inner peripheral surface 23 of the clip 20, such that the insertion angle of the anchoring screw 30 may be adjusted. Therefore, according to the present invention, as described above, the procedure may be performed while variably adjusting the insertion position of the anchoring screw 30, and the insertion angle at which the anchoring screw 30 is inserted into the bone may be appropriately adjusted depending on a state of a patient's diseased part.

In addition, FIG. 9 is a cross-sectional view of another embodiment in which the bone anchoring device according to the present invention is applied. Referring to FIG. 8, the head part 31 of the anchoring screw 30 may be configured as a locking screw having an inclined side surface having a cross-section with a width that decreases from above to below. Therefore, the inner peripheral surface 23 of the clip 20 may be an inclined surface having an inclination and a diameter that decreases from above to below while corresponding to the head part 31.

In this case, a fixing screw thread may be formed on the outer peripheral surface 33 of the head part 31 and configured to be screw-coupled to the inner peripheral surface 23 of the clip 20. An inner screw thread may be formed on the inner peripheral surface 23 of the clip 20 while corresponding to the fixing screw thread. Therefore, according to the present invention, the anchoring screw 30 may be more securely fixed to the plate 10, thereby providing a strong fixing force to the fractured bone.

In addition, a screw (cortex screw) having cut-out grooves formed at predetermined intervals may be formed in a lateral surface of the head part 31. Therefore, according to the present invention, it is possible to partially adjust an angle of the screw inserted into the bone while providing a strong fixing force to the fractured bone.

As described above, according to the present invention, the inner peripheral surface 23 of the clip 20 is manufactured to correspond in shape to the head part 31 of the anchoring screw 30, such that various screws may be applied to the single plate 10 without separately manufacturing the plates 10 for the respective screws to be used, which makes it possible to improve compatibility between the bone anchoring components.

In addition, according to the present invention, although not illustrated in the drawings, the screw thread 34 of the body part 32 may include: an insertion thread portion formed at an end of the body part 34; and an extraction thread portion formed between the insertion thread portion and the head part and configured to provide a drawing force to the anchoring screw 30 by being screw-coupled to the bone and rotated in case of drawing and removing the anchoring screw 30.

In this case, a screw thread pitch of the extraction thread portion may be larger than a screw thread pitch of the insertion thread portion. Therefore, the anchoring screw 30 is not coupled to the bone when the anchoring screw 30 is inserted. The anchoring screw 30 is screw-coupled to the bone only when the anchoring screw 30 is removed after the bone coalesces.

In addition, according to the present invention, a drill tip is formed at an end of the insertion thread portion to drill the bone in case of inserting the anchoring screw 30 into the bone, such that the anchoring screw 30 may be more easily inserted into the bone.

While the exemplary embodiments of the present invention have been described above, those skilled in the art may variously modify and change the present invention by adding, changing, deleting or modifying constituent elements without departing from the spirit of the present invention disclosed in the claims, and the modification and change also belong to the scope of the present invention.

For example, each component described as a single type may be carried out in a distributed manner. Likewise, components described as a distributed type can be carried out in a combined type. The scope of the present invention is represented by the claims to be described below rather than the detailed description, and it should be interpreted that the meaning and scope of the claims and all the changes or modified forms derived from the equivalent concepts thereto fall within the scope of the present invention.

### INDUSTRIAL APPLICABILITY

The present invention relates to the bone anchoring device in which the rectangular fastening hole may be formed in the plate configured to come into close contact with the fractured bone, the clip may be inserted into the fastening hole, and the clip may slip in the transverse direction in the fastening hole, such that the position of the bone anchoring screw for fixing the bone may be adjusted. According to the present invention, it is possible to adjust the position and insertion angle of the bone anchoring screw and improve the compatibility between the bone anchoring components.

1. A bone anchoring device, which anchors a fractured bone by fixing a plate being in close contact with a fractured bone by using a screw, the bone anchoring device comprising:
a plate configured to be attached to the fractured bone and having a plurality of fastening holes to support the fractured bone, wherein at least one of the fastening holes is formed in a rectangular shape having an opening portion elongated in a longitudinal direction;
a clip configured to be inserted into the rectangular fastening hole and capable of slipping in the longitudinal direction in the rectangular fastening hole; and
an anchoring screw configured to be fastened into the clip and fastened into the bone.

2. The bone anchoring device of claim 1, wherein the rectangular fastening hole is formed in the longitudinal direction of the plate.

3. The bone anchoring device of claim 1, wherein the clip has an elastic structure having a cut-out portion, the clip is inserted into the rectangular fastening hole while being elastically compressed, and the separation of the clip from the rectangular fastening hole is restricted as the clip is elastically deformed after the clip is inserted into the rectangular fastening hole.

4. The bone anchoring device of claim 3, wherein an outer peripheral surface of the clip is formed as a curved surface convex outward, and an inner surface of the rectangular fastening hole based on the longitudinal direction is formed as a curved surface concave inward that corresponds to the outer peripheral surface of the clip.

5. The bone anchoring device of claim 4, wherein a guide protrusion protrudes outward from the outer peripheral surface of the clip, a guide groove is recessed inward in the inner surface of the rectangular fastening hole based on the longitudinal direction at a position corresponding to the guide protrusion, the clip slips only in a horizontal direction, and angular deformation and a rotation are restricted.

6. The bone anchoring device of claim 5, wherein the guide protrusion is formed in a polyhedral shape.

7. The bone anchoring device of claim 6, wherein an edge of the guide protrusion is formed in a curved shape.

8. The bone anchoring device of claim 3, wherein an outer peripheral surface of the clip is formed as a flat surface that adjoins an inner surface of the rectangular fastening hole in the longitudinal direction, and the rectangular fastening hole has a flat surface that is concave inward so that the inner surface based on the longitudinal direction corresponds to the outer peripheral surface of the clip.

9. The bone anchoring device of claim 1, wherein the anchoring screw comprises:
a head part configured to be seated on an inner peripheral surface of the clip; and
a body part extending from an inner end of the head part, configured to come into contact with the bone, and having a screw thread formed on an outer peripheral surface thereof.

10. The bone anchoring device of claim 9, wherein the head part is configured as a motion screw having an inclined curved surface having a cross-section with a width that decreases downward, and the inner peripheral surface of the clip has an inclined curved surface having a diameter that decreases downward while corresponding to the head part.

11. The bone anchoring device of claim 9, wherein the head part is configured as a locking screw having an inclined side surface having a cross-section with a width that decreases from above to below, and the inner peripheral surface of the clip is an inclined surface having an inclination and a diameter that decreases from above to below while corresponding to the head part.

12. The bone anchoring device of claim 11, wherein a cortex screw having cut-out grooves formed at predetermined intervals is formed in a lateral surface of the head part.

13. The bone anchoring device of claim 11, wherein a fixing screw thread is formed on an outer peripheral surface of the head part and configured to be screw-coupled to the inner peripheral surface of the clip, and an inner screw thread is formed on the inner peripheral surface of the clip while corresponding to the fixing screw thread.

14. The bone anchoring device of claim 9, wherein the screw thread of the body part includes:
an insertion thread portion formed at an end of the body part; and
an extraction thread portion formed between the insertion thread portion and the head part and configured to provide a drawing force to the anchoring screw by being screw-coupled to the bone and rotated in case of drawing and removing the anchoring screw.

15. The bone anchoring device of claim 14, wherein a screw thread pitch of the extraction thread portion is larger than a screw thread pitch of the insertion thread portion.

16. The bone anchoring device of claim 14, wherein a drill tip is formed at an end of the insertion thread portion to drill the bone in case of inserting the anchoring screw into the bone.
